(19) United States
(12) Patent Application Publication
(10) Pub. No.: US 2025/0044307 A1
(43) Pub. Date: Feb. 6, 2025

(54) Quality Control Materials for Cardiac Troponin Testing

(71) Applicant: McMaster University, Hamilton (CA)

(72) Inventors: Peter Kavsak, Burlington (CA); Lorna Clark, Hamilton (CA)

(21) Appl. No.: 18/713,411

(22) PCT Filed: Nov. 25, 2022
(86) PCT No.: PCT/CA2022/051733

§ 371 Date: May 24, 2024

Related U.S. Application Data
(60) Provisional application No. 63/283,366, filed on Nov. 26, 2021.

(51) Int. Cl. G01N 33/96

(52) U.S. Cl. CPC G01N 33/96; G01N 2496/05

(57) ABSTRACT

A method for the preparation of a cardiac troponin quality control sample of a mammal to determine the accuracy and/or precision of a cardiac troponin assay over time is provided. The method comprises the steps of: i) preparing a cardiac troponin concentrate from a pool of biological samples obtained from a population of the mammal having a troponin concentration of greater than 75% of an upper analytical limit of the assay; ii) obtaining a cardiac troponin base material comprising whole blood, plasma or serum of the mammal which is interference-free and disease-free; and iii) combining a quantity of the troponin concentrate with the base material to yield a quality control sample having a target concentration. Quality control samples may be provided in kits which provide a set of samples having particular concentrations which essentially correspond with lower detectable limits of an assay, upper limits of normal, as well as concentrations which define risk stratification cutoffs and other diagnostic cutoffs.

### FIELD

The present application relates to construction of quality control materials for use to monitor and assess accuracy of laboratory tests and, in particular, to quality control samples for use in cardiac troponin (cTn) testing, as well as methods of making and using such samples to determine precision and accuracy of assays and instruments used by laboratories who provide testing for cardiac troponin.

### BACKGROUND

Proper monitoring for cardiac troponin (e.g. sensitive and high-sensitivity cardiac troponin (hs-cTn) testing) requires precision and accuracy testing at the lower limits of detection, as well as at cardiac troponin decision levels.

Laboratories require quality control (QC) materials as part of quality assurance (QA) so that objective evidence is obtained to confirm that a test is analytically correct (i.e., accurate and precise). For cardiac troponin testing having the correct QC materials at appropriate concentrations or levels to test assays and instruments are vital for proper reporting and interpreting.

One component that defines a high-sensitivity cardiac troponin (hs-cTn) assay is achieving a 10% coefficient of variation (CV) at the 99th-percentile concentration of a healthy population. Often, this imprecision is assessed at the overall population's 99th-percentile concentration (i.e., a cutoff derived from both healthy females and healthy males). However, as sex-specific 99th-percentiles are recommended by clinical and laboratory guidelines, it is imperative that the imprecision is also assessed at the lower female 99th-percentile concentration given this cutoff is used to identify myocardial injury in the female population.

For these purposes quality control materials to test the lower limit of reporting, normal levels, the sex-specific 99th percentile upper limits of normal from a healthy population, other population upper limits of normal, risk stratification cutoffs and other diagnostic cutoffs such as those used in the Clinical Chemistry Score and the 0-1 h, 0-2 h, 0-3 h algorithms, High-STEACS and COMPASS-MI algorithms, would be valuable for laboratories, clinicians, and patients.

Thus, it would be desirable to develop suitable materials for laboratories, clinicians, and patients to use to determine the accuracy and/or precision of cardiac troponin concentration measurements. Providing materials that provide objective evidence that cardiac troponin testing is analytically acceptable would enable laboratories to confidently report results to clinicians for early diagnosis of injury and for risk stratification in patients, and to provide confidence to patients that perform their own cardiac troponin testing.

### SUMMARY

Herein, a novel protocol and kit for quality control (QC) materials is provided to determine the precision and/or accuracy of cardiac troponin assays and analyzers to quantify cardiac troponin, especially for high-sensitivity cardiac troponin (hs-cTn) assays at selected levels, which aid to minimize test error and potentially patient harm as a result of inaccurate assay results. The protocol yields accurate, reproducible and stable QC material to be used alongside cardiac troponin/hs-cTn testing.

Thus, in one aspect of the invention, a method for the preparation of a quality control sample of a mammal for use with a given cardiac troponin assay is provided comprising the steps of:
i) preparing a cardiac troponin concentrate from a pool of biological samples obtained from a population of the mammal having a troponin concentration of greater than 75% of an upper analytical limit of the assay;
ii) obtaining a cardiac troponin base material comprising whole blood, plasma or serum of the mammal which is disease-free; and
iii) combining a quantity of the troponin concentrate with the base material to yield a quality control sample having a target concentration.

In another aspect of the invention, novel troponin quality control samples are provided.

In a further aspect of the invention, a kit is provided comprising quality control samples prepared using the methods herein described.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description.

### DETAILED DESCRIPTION

A method for preparing cardiac troponin quality control sample of a mammal for use with a given cardiac troponin assay is provided. The method comprises the steps of: i) preparing a cardiac troponin concentrate from a pool of biological samples obtained from a population of the mammal having a troponin concentration of greater than 75% of an upper analytical limit of the assay; ii) obtaining a cardiac troponin base material comprising whole blood, plasma or serum of the mammal which is interference-free and disease-free; and iii) combining a quantity of the troponin concentrate with the base material to yield a quality control sample having a target concentration.

In a first step of the present method, a cardiac troponin concentrate is prepared from a pool of mammalian biological samples having troponin concentrations of greater than 75% of an upper analytical limit of a given assay for cardiac troponin detection. As one of skill in the art will appreciate, the concentration of cardiac troponin I or T in a suitable biological sample may be determined using immunoassay methodology (i.e., based on ELISA principles of sandwich or competitive immunoassays). These assays may be sensitive cardiac troponin (cTn) assays or high sensitivity cardiac troponin (hs-cTn) assays. Hs-cTn assays are herein defined as assays able to detect cTn in the single digit range of nanograms per litre, e.g., measurable concentrations less than 10 ng/L, with a coefficient of variation (CV) of ≤10% of the 99th percentile of cTn concentration in reference subjects (the recommended upper reference limit [URL]), and measurable concentrations of cardiac troponin in greater than 50% of a general population. Sensitive-cTn assays exhibit a CV of <20% at the 99th percentile URL, and measurable concentrations of cardiac troponin in a general population of less than 50%. Thus, the upper analytical limit, or upper limit of quantification is the highest concentration troponin that can be determined using a given analytical assay with the required precision and accuracy. Concentrations above the upper analytical limit are reported as “greater than” the upper analytical limit and require a dilution to provide an actual number.

Precision is a measure of reproducibility or repeatability of a measurement. If the same sample is measured multiple times, the analytical assay may provide slightly different values (i.e. concentrations). Generally, validated assays have an expected precision less than or equal to ±15% at all concentrations. Precision may be determined by making at least 3 measurements of the same sample. The coefficient of variation (% CV) is calculated by dividing the standard deviation of the 3 measurements by the mean of the 3 measurements and multiplying by 100.

Accuracy is a measure of how close a measured value is to the actual (true) value. To determine the accuracy of an assay, a known concentration (such as that of a quality control sample of the invention) is measured multiple times, and the accuracy of the assay is the difference between the average of the measured concentrations and the known concentration divided by the known concentration.

Suitable biological samples may include whole blood, serum, plasma, pericardial fluid samples, or cardiac tissue. Preferred biological samples include whole blood, serum or plasma samples. The concentrate may comprise cardiac troponin I (cTnI) or troponin T (cTnT) or a combination thereof (e.g. cardiac troponin I and cardiac troponin T may be present together in the presence of troponin C). Generally suitable biological samples will have a cTn concentration approaching the upper analytical limit of a selected assay, for example, at least 75% of the upper analytical limit, preferably at least 80-96% of the upper analytical limit. Thus, the cardiac troponin I concentration of the selected biological samples may be at least about 20,000 ng/L, preferably at least about 30,000 ng/L or 40,000 ng/L, as determined using a high sensitivity troponin I assay. For example, for the Abbott ARCHITECT hs-cTnI assay, selected biological samples may comprise cTnI within the range of about 40000 ng/L to 48000 ng/L which is within 80-96% of an upper analytical detection limit (e.g. about 50,000 ng/L). For cardiac troponin T, as it is a different protein detected using a different assay, the upper analytical limit is different, e.g. about 10,000 ng/L. Thus, 80-96% of the upper analytical limit for cTnT biological samples is a concentration in the range of about 8000-9600 ng/L.

The biological sample is processed to provide a cTn concentrate. In one embodiment, a fluid sample such as blood, serum, plasma and/or pericardial fluid, is used to prepare the cTn concentrate. The sample is processed to remove cellular debris, interferences such as lipids, free hemoglobin, bilirubin, globulins, antibodies such as human anti-mouse antibodies (HAMAs) and drugs, and macrocomplexes such as protein complexes. An anticoagulant is added, e.g. for plasma, to remove some interferences. In this regard, a preferred anticoagulant is EDTA (ethylenediaminetetraacetic acid). Cellular debris and clot material may be removed by centrifugation under suitable conditions such as ≥1500 g-force (RCF) for about 15 minutes or 3000 RCF for about 10 minutes. The sample fluid, such as plasma or serum, is then removed from the cellular debris and clot material. The plasma/serum is tested for presence of macrocomplexes, interferences, and infectious disease (i.e., HIV and other viral and/or bacterial infection), and processed further to remove any such contaminating agents using techniques established in the art such as ultrafiltration and chromatographic techniques such as ion exchange, gel filtration and affinity resins. If interferences cannot be removed or samples have tested positive for infectious disease, then the samples will be discarded and a new collection process will begin. The processed fluid samples are then pooled and may be stored by freezing (below −20° C.) prior to use.

If cardiac tissue is used, the tissue is homogenized on ice under non-denaturing conditions in buffer containing protease and phosphatase inhibitors. The homogenate is then centrifuged to separate solids from liquids (e.g. for 10 min at 16 000 g and 4° C.). The supernatant may be further processed to remove any other potential interferences using filtration and/or chromatographic methods, and tested for infectious disease. A clean fluid sample derived from cardiac tissue and free from infectious disease results which may be utilized as a cardiac troponin concentrate for quality control sample preparation.

A base material comprising mammalian whole blood, plasma and/or serum, optionally including a diluent (e.g. phosphate-buffered saline), which is suitable for troponin testing, is also utilized in the present method. The base material functions to provide stability to the quality control samples, and thereby, facilitates or supports the accuracy of the quality control samples. The base material is processed to remove cellular debris and other contaminants (e.g. interferences and macrocomplexes), for example, by filtration and/or centrifugation, if required. Plasma for use in the method is treated with a coagulating agent such as CPD (citrate phosphate dextrose), ACD (citrate dextrose) or other suitable coagulating agent to remove coagulating proteins. The base material for use in the method will be disease-free. If the base material is human-derived, it may be prepared from any blood type. In one embodiment, the base material is prepared from human blood type AB, which may be RhD positive, RhD negative or a combination thereof. AB blood unexpectedly and advantageously provides a very stable base material, with little or no complex formation over time, and as such yields quality control samples that provide enhanced accuracy. The base-material may be stored frozen (below −20° C.) prior to use.

If frozen, the cTn concentrate and/or base-material are allowed to thaw overnight at 2-8° C. (e.g. in a refrigerator) and are then warmed at room temperature prior to mixing. For mixing, the tube of concentrate or base material is inverted at least 5 times.

The pooled cTn concentrate and base materials are separately tested, for example in triplicate, to obtain their cTn value or concentration. It may be preferable to determine the cTn value of the concentrate and base material using each sensitive cardiac troponin (cTn) assay and/or high sensitivity cardiac troponin (hs-cTn) assay for which the quality control samples are being made. A number of such assays have been developed. Examples include, but are not limited to, hs-cTn assays developed by Abbott Laboratories (Abbott), Beckman Coulter (Beckman), Hoffman-La Roche Ltd (Roche), QuidelOrtho, and Siemens Healthineers (Siemens). Determination of the cTn concentration using the assay for which the QC samples are being prepared will assist in more accurate determination of the amounts of cTn concentrate and base material to be combined in the preparation of quality control samples of selected concentrations for use in each assay.

High sensitivity cTnI assays include assays made by Abbott, Siemens QuidelOrtho, and Beckman. For example, the Abbott ARCHITECT STAT™ Troponin-I assay, Siemens Atellica™/Advia™ High Sensitivity Troponin I assays and Beckman Access hs-cTnI assay are each similarly a two-step immunoassay that detects cTnI in a human biological sample using chemiluminescent technology. In the first step, the sample is combined with anti-troponin-I antibodies coated with paramagnetic microparticles to bind or capture cTnI present in the sample. A pair of monoclonal antibodies are utilized which are directed against epitopes in the heart-specific and the stable region of the troponin I molecule close to the amino (NH2) terminus (i.e. epitopes at amino acid positions, 24-40 and 41-49, of troponin I). In the second step, anti-troponin-I labeled conjugate (such as an acridinium-labeled conjugate as used in the Abbott assay) is added to the sample and reacts with the bound microparticles to result in a detectable chemiluminescent reaction by which to detect captured troponin I.

Sensitive and high sensitivity cTnT assays include assays made by Roche. For example, the sensitive CARDIAC POC troponin T assay is an immunoassay which utilizes two fragment antigen-binding (FAB) of two cTnT-specific monoclonal antibodies, a gold-labelled signal antibody and a biotinylated antibody, in a sandwich format. The antibodies recognize epitopes located in the central part of the cTnT molecule (amino acid positions 125-131 and 135-147). The cTnT sandwich complex is then conjugated to streptavidin-coded magnetic microparticles, which are captured and gold-labelled cTnT sandwich complexes are detected by electrochemiluminescence, the intensity of which correlates with troponin T concentration.

To make the quality control (QC) samples, the cTn concentrate is added to the base-material in amounts calculated to yield quality control samples of particular target concentrations, i.e. concentrations which are specific for use with a given cTn assay and/or analyzer. Quality control sample concentrations will generally be within 1.6 ng/L or 20% of a target concentration.

In another aspect, the present invention provides a kit of quality control samples for use with given cTn assays and algorithms which provide a set of quality control samples comprising target concentrations for analysis with select assays, analyzers and/or algorithms at regular intervals. The quality control samples are used with the assay to confirm that the results obtained (i.e. cTn concentrations) correspond with the previously determined cTn concentrations of each quality control sample. In this way, accuracy of the assay to analyze the target concentrations is confirmed on a regular basis, e.g. daily, weekly, monthly, bi-monthly, quarterly or semi-annually, using the quality control samples. Optimal precision of the assay is confirmed when results yield an acceptable standard deviation of less than 0.8 ng/L and/or coefficient of variation, i.e. ≤10%. Accuracy is confirm if the concentration is within 1.6 ng/: or 20% of the target concentration. For the purpose of accuracy, the concentration of each quality control sample may be determined once, or preferably, will be based on the average concentration of 3 or more determinations.

The kit will generally include quality control samples having target concentrations which are relevant to the assay/analyzer, algorithm and/or setting within which the assay is being used. The kit includes at least a lower limit of detection quality control sample having a target concentration of about 2 ng/L greater than the lower limit of detection of a selected troponin assay. The lower limit of detection is the lowest concentration which can be measured analytically using a given troponin assay. In one embodiment, the lower limit of detection is a bioanalytical response that is five times the background response in an assay. Alternatively, the lower limit of detection as the limit of blank, limit of detection, limit of quantitation, or lower linearity limit, each of which is known in the art.

The kit may also include one or more quality control samples as follows:
i) a quality control sample which is about 4 ng/L greater than the lower limit of detection quality control sample to confirm that differences of 4 ng/L are detectable;
ii) an upper limit of normal quality control sample at about the 99^{th} percentile of a normal population;
iii) a quality control sample having a target concentration that is at about a sex-specific cut-off in cardiac myocardial infarction diagnosis;
iv) one or more quality control samples having a target concentration that define myocardial infarction risk stratification cutoff concentrations;
v) a quality control sample having a target concentration that is prognostic in non-cardiac surgery; and
vi) a quality control sample having a target concentration that is prognostic in cardiac surgery.

In one embodiment, a QC sample for use with an hs-cTnI assay, e.g. an Abbott hs-cTnI assay, is prepared. The lower limit of detection/reporting for hs-cTnI assays ranges from about 1 to about 4 ng/L. As one of skill in the art will appreciate, this lower limit is dependent on the assay used, different laboratory practices and variable regulatory requirements. Accordingly, the target concentration for an appropriate QC sample for use with an hs-cTnI assay is about 6 ng/L (˜2 ng/L higher than the highest listed lower limit from the range).

In another embodiment, a QC sample for use with an hs-cTnT assay, e.g. a Roche hs-cTnT assay, is prepared. Based on the highest value of the lower limit of reporting, the target concentration for an appropriate QC sample for use with an hs-cTnT assay is about 8 ng/L (˜2 ng/L higher than the highest listed lower limit (6 ng/L) from the range).

In another embodiment, QC samples for use with a 0/1-hour, 0/2-hour or 0.3-hour algorithm is prepared. A 0/1-hour, 0/2-hour or 0.3-hour algorithm is employed which identifies or rules out myocardial infarction using cardiac troponin levels determined on presentation (0) and then again at 1, 2 or 3 hours following presentation, along with clinical presentation. The 0/1 and 0/2 algorithms triage patients by applying assay-specific cardiac troponin thresholds lower than the 99th percentile of a normal reference population at presentation along with absolute changes within the first hour or second hour. The 0/3 algorithm threshold is based on the 99th percentile of a normal reference population at presentation and at the 3-hour mark. Thus, quality control samples are based on the thresholds in each algorithm. The 0/1 hour algorithm using a hs-cTnI assay, such as the Abbott hs-cTnI assay, lists 4 ng/L, 5 ng/L, and 64 ng/L as important cutoffs, while the 0/2 hour algorithm lists 4, ng/L, 6 ng/L, and 64 ng/L as cut-offs. The 0/3 hour algorithm lists 16 ng/L, 26 ng/L, and 34 ng/L as cutoffs. Accordingly, a kit providing quality control samples for any one of these algorithms is provided comprising QC samples with cut-off concentrations for the selected algorithm. A combined kit may also be provided comprising QC samples of 4 ng/L, 6 ng/L, 16 ng/L, 30 ng/L and 60 ng/L for use by laboratories that use the 0/1 hr, 0/2 hr and/or 0/3 hr algorithms with an hs-cTnI assay. In this regard, it is noted that 30 ng/L is within +/−20% of 26 ng/L and 34 ng/L, and 60 ng/L is within +/−20% of 64 ng/L. For an hs-cTnT assay, a kit comprising quality control samples having concentrations of 7 ng/L, 10 ng/L, 15 ng/L, 20 ng/L and 50 ng/L are appropriate to confirm accuracy when using the 0/1 h, 0/2 h and/or 0/3 h algorithm.

The COMPASS-MI algorithm is a tool that utilizes high-sensitivity cTnI or cTnT assays to measure an initial cutoff, early/late delta troponin, and then calculates the diagnosis depending in the combination of assay, cutoff, and delta timing. The COMPASS-MI algorithm for a hs-cTnI assay is optimized when using a cutoff of 4 ng/L with a difference of less than 4 ng/L, and a cutoff of 60 ng/L with a difference of at least 18 ng/L. Accordingly, a kit comprising quality control samples of 4 ng/L, 8 ng/L, 40 ng/L and 60 ng/L would be suitable for use with the COMPASS-MI algorithm for a hs-cTnI assay such as the Abbott assay. For use of the COMPASS-MI algorithm with a hs-cTnT assay such as the Roche assay, quality control samples having concentrations of 7 ng/L, 11 ng/L, 55 ng/L and 70 ng/L are appropriate for similar reasons with cutoffs of 7 ng/L and 70 ng/L.

The Clinical Chemistry Score (CCS) is a laboratory-based risk-stratification tool that combines the determination of hs-cTn with glucose and estimated glomerular filtration rate, to identify patients at risk of myocardial infarction or death. The CCS utilizes cTnI concentrations of 4 ng/L, 14 ng/L and 30 ng/L. Thus, a kit comprising QC samples having these target concentrations within 1.6 ng/L or 20% would be appropriate. For cTnT, the CCS utilizes concentrations of 8 ng/L, 18 ng/L and 30 ng/L. Thus, a kit comprising QC samples having these target concentrations within 1.6 ng/L or 20% would be appropriate.

High-Sensitivity Troponin in the Evaluation of patients with Acute Coronary Syndrome (High-STEACS) incorporates a high-sensitivity cardiac troponin I threshold of 5 ng/L. Patients without myocardial ischaemia according to ECG results and cardiac troponin concentrations <5 ng/L at presentation are considered low risk. Patients with cardiac troponin concentrations ≥5 ng/L at presentation are retested at 3 hours after presentation and ruled out at 3 hours if cardiac troponin concentrations are unchanged (delta <3 ng/L) and remain ≤99th percentile. Thus, concentrations for use with the High-STEACS pathway using a high sensitivity cTnI assay include 5 ng/L, 16 ng/L (for females, 99^{th} percentile using the Abbott hs-cTnI); and 34 ng/L (for males, 99^{th} percentile using the Abbott hs-cTnI). Accordingly, a kit comprising quality control samples for use with the High-STEACS pathway having target cTnI concentrations of 5 ng/L, 9 ng/L (to account for a delta of greater than 3 ng/L), 16 ng/L and 34 ng/L is suitable. Similarly, quality control samples for use with the High-STEACS pathway using a high sensitivity cTnT (hs-cTnT) assay includes QC samples having target troponin T concentrations of 6 ng/L, 10 ng/L, 14 ng/L and 22 ng/L.

Quality control in perioperative and/or out-patient monitoring is also important. In this case, quality control samples for use with a hs-cTnI assay may include cTnI concentrations of 6 ng/L, 10 ng/L, 15 ng/L, 30 ng/L, 60 ng/L, 700 ng/L and/or 2600 ng/L. The 6 ng/L and 10 ng/L concentrations are useful to identify low troponin concentrations and confirm that the lab can measure differences of at least 4 ng/L. The 15 ng/L and 30 ng/L concentrations are relevant to sex-specific cutoffs (within +/−20%). The 60 ng/L and 700 ng/L concentrations are cutoffs that have prognostic importance in non-cardiac surgery, and the 2600 ng/L concentration is useful in cardiac surgery as values that are at least 100-fold higher than the overall 99^{th} percentile are important for risk stratification. Similarly, quality control samples for use with a hs-cTnT assay may include cTnT concentrations of 7 ng/L, 10 ng/L, 14 ng/L, 20 ng/L, 65 ng/L, 1000 ng/L and 2800 ng/L.

A laboratory compliant kit is also provided. In one embodiment, the kit comprises quality control samples having cTnI concentrations of 8 ng/L, 18 ng/L, 36 ng/L and 300 ng/L. This is suitable for use with a hs-cTnI assay. The 8 ng/L sample is relevant since it is at least 4 ng/L higher than the lowest reportable limit. The 18 ng/L sample is at least 10 ng/L higher than the first sample, and the 36 ng/L sample is at least double the second sample. The 300 ng/L sample is relevant as it is equivalent to the prior World Health Organization (WHO) cutoff for cardiac troponin using the Abbott assay. Similarly, for use with an hs-cTnT assay, cTnT quality control samples in a laboratory compliant kit include concentrations of 8 ng/L, 18 ng/L, 50 ng/L and 100 ng/L (with 100 ng/L being equivalent to the prior WHO cutoff using the Roche cTnT assay).

Quality control samples are also useful for machine learning (ML) based and artificial intelligence (AI) algorithms. In this regard, quality control samples having troponin concentrations of 8 ng/L, 16 ng/L, 30 ng/L, 60 ng/L, 100 ng/L and/or 200 ng/L represent concentrations that may be used in various algorithms that use troponin as a continuous variable. While the content of the kit may vary, a kit comprising each of the above six quality control samples covers the relevant range of concentrations which are appropriate for both hs-cTnI and hs-cTnT.

As one of skill in the art will appreciate, the quality control samples and kits identified above for use with a given algorithm are suitable for use with any high-sensitivity cardiac troponin I assay or high-sensitivity cardiac troponin T assay.

The kit may additionally comprise instructions for care of the quality control samples and/or for use of the quality control samples.

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±20% of the modified term if this deviation would not negate the meaning of the word it modifies.

As used in this application, the singular forms “a”, “an” and “the” include plural references unless the content clearly dictates otherwise. For example, an embodiment including “Quality Control” should be understood to present certain aspects with one substance or two or more additional substances.

In embodiments comprising an “additional” or “second” component, such as an additional or second Quality Control level, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

### EXAMPLES

The following non-limiting examples are illustrative of the present application.

### Example 1-Method of Preparing Quality Control Samples to Monitor a Hs-cTnI Assay

A high concentration cTnI sample was prepared for use in an Abbott hs-cTnI assay. Ethylenediamine tetraacetic acid (EDTA) plasma samples having cTn concentrations near the upper analytical limit (such as within 80% to 96% of the upper analytical range limit) were pooled. Thus, for the Abbott ARCHITECT hs-cTnI assay selected samples ideally comprise a concentration of cTnI of 40000 ng/L to 48000 ng/L (80% to 96% of 50000 ng/L, the upper analytical range limit for this assay).

The high concentration hs-cTnI pooled sample (i.e., the high concentrate) was centrifuged (with a force ≥1500 RCF for about 15 minutes) to remove any debris or cellular material. The resulting concentrate was determined to be free of macrocomplexes and no infectious agents. The plasma was added to a plastic (polypropylene) container and frozen (below −20° C.).

A base material was prepared from citrate phosphate dextrose (CPD) plasma from type AB blood and frozen (below −20° C.).

The concentrate and base material were allowed to thaw and warmed to room temperature prior to mixing. For mixing, the concentrate and base-material were inverted at least 5 times. The base material was filtered to remove debris.

Each of the high concentrate and base material were tested in triplicate to obtain a cTnI concentration for each.

Based on the cTnI concentrations of the high concentrate and base material, a suitable volume of the high concentrate was added to the base material to provide a quality control sample at a concentration of approximately 5 ng/L for use in the Abbott ARCHITECT hs-cTnI assay. The concentration was selected to be within 1.6 ng/L or 20% of 6 ng/L, a target concentration adjusted up by 2 ng/L from the lower limit of detection of 4 ng/L using the Abbott assay.

This quality control sample was tested in one laboratory when comparing different lots of Abbott hs-cTnI reagents and calibrators over 1439 days (17 different comparisons consisting of 73 tests in total). The average concentration was determined to be 5.1 ng/L across the course of testing with a standard deviation of 0.6 ng/L, confirming acceptable performance of the QC sample at the lower limit of detection.

Thus, the QC sample confirmed the accuracy of Abbott hs-cTnI testing in this laboratory for nearly 4 years while also demonstrating acceptable precision (i.e., SD≤0.8 ng/L).

### Example 2

A multi-site ‘CODE-MI’ sub-study was conducted to assess the hs-cTn assay imprecision at the female 99th-percentile concentration for different hs-cTn assays.

Patient samples (EDTA plasma) with cTn concentrations near the upper analytical limit of the assay were identified and pooled together to form the high cTn concentrate. Human serum from AB blood type was purchased from Sigma-Aldrich and used as the base material into which the high cTn pool was spiked to produce different quality control (QC) samples. For each QC sample, the concentration was derived from testing the spiked serum ten times, with the first and last aliquot tested prior to freezing the QC aliquots below −70° C. Two aliquots were thawed and retested (to confirm stability) prior to sending the QC aliquots on dry ice to laboratories across Canada.

The analyzers used in the study were the Abbott ARCHITECT i1000 (hs-cTnI), Beckman Access 2 (hs-cTnI), Roche Cobas 602 (hs-cTnT), and Siemens ADVIA Centaur (hs-cTnI) for the initial assignment of QC hs-cTn concentrations targeted slightly below the manufacturers' female 99th-percentile hs-cTn concentrations (average concentration of QC sample concentrations under the United States 99th-percentile concentration [ng/L]: Abbott=14.1|16; Beckman=9.7|12; Roche=8.8|9; Siemens=36.6|39).

A summary of the process used to prepare the QC sample for the Abbot assay is provided in Table 1.

TABLE 1

| Step 1 - base | Millipore/Sigma serum base H6914 (infectious | |
|---|---|---|
| material | agent free, AB clotted whole blood type) was | |
| | purchased as 5 × 100 mL bottles and stored | |
| | at −20° C. until needed | |
| Step 2 - base | Serum thawed at 4° C. overnight, placed at room | |
| material thawed | temp. to warm up, and mixed by swirling bottle | |
| Step 3 - base | Serum was tested three times with the Abbott | |
| material tested | hs-cTnI assay and yielded results of 2.29, | |
| for hs-cTnI assay | 2.29, 2.05 ng/L (CV = 6.27%) | |
| Step 4 - high | Frozen high concentrate patient pool (infectious | |
| concentrate | agent free), was thawed, centrifuged for 10 min | |
| tested | at 3000 g, and tested 3 times with the Abbott hs- | |
| | cTnI assay to yield results of 40383.66, 43088.02, | |
| | 42560.22 ng/L (CV = 3.41%) | |
| Step 5-14 ng/L | 35 uL of high cTn concentrate was added to about | |
| material made | 99 mL of base to get a target value of about 14 | |
| | ng/L. Spiked material was poured into a beaker, | |
| | and stirred for 10 mins @ ~300 rpm. The sample | |
| | was tested 10 times with the Abbott hs-cTnI assay: | |
| | | QC (ng/L) |
| | 1 | 13.95 |
| | 2 | 13.91 |
| | 3 | 14.94 |
| | 4 | 13.43 |
| | 5 | 14.17 |
| | 6 | 14.22 |
| | 7 | 14.79 |
| | 8 | 14.18 |
| | 9 | 14.13 |
| | 10 | 13.73 |
| | average | 14.15 |
| | SD | 0.45 |
| | CV | 3.18% |

Instructions on storing, handling, and testing using the quality control samples were provided to the laboratories. The QC samples were transported frozen on dry ice, with the recipient laboratories storing the frozen QC samples/materials at −200° C. or colder (preferably below −700° C.). Once a month, QC material was used for testing. The QC material was allowed to thaw on the bench at room temperature for 15 minutes. QC material was not to be thawed in a water bath, nor warmed by hands. After 15 minutes, the samples were mixed to confirm material is thoroughly thawed and then the material was centrifuged (3000×g for 10 minutes). The QC sample was pipetted into an appropriately labelled sample cup with the frequency of testing being once a month for one year. Data obtained from the first three months for assays that had at least ten data points was used to determine the pooled SDs and weighted mean used to calculate the overall CV for each company.

Testing of 168 samples occurred on 57 different instruments from 35 sites across 8 provinces from January 2022 to March 2022. Instruments included the Abbott ARCHITECT i1000 (n=1), i2000 (n=5), Alinity (n=2); Beckman Access 2 (n=1), DxI 600 (n=3), DxI 800 (n=5); Roche Cobas e411 (n=9), e601 (n=4), e602 (n=12) e801 (n=11); and Siemens Atellica (n=4). The range of QC material concentrations were 10.7-15.6 ng/L for Abbott (n=24); 9.3-13.5 ng/L for Beckman (n=25); 3.7-11.5 ng/L for Roche (n=107); and 29.2-37.0 ng/L for Siemens (n=12). The overall average hs-cTn concentrations (CV) were: Abbott=12.7 ng/L (7%); Beckman=11.6 ng/L (7%); Roche=8.6 ng/L (10%); Siemens=33.4 ng/L (5%).

The initial estimates from this analytical sub-study of the CODE-MI trial indicate acceptable, accurate and comparable precision of hs-cTn assays at the female-specific 99th-percentile concentration cutoff.

### Example 3-Summary of QC Sample Kits for Use with Different cTn Algorithms and Assays

Table 2 provides a summary of QC sample kits for use with different cTn algorithms and assays.

TABLE 2

| QC Kit | Abbott hs-cTnI | Roche hs-cTnT |
|---|---|---|
| Lower Limit (2 ng/L higher | 6 ng/L | 8 ng/L |
| than lowest limit reported in | | |
| the United States) | | |
| 0/1 h, 0/2 h, 0/3 h algorithms | 4 ng/L, 6 ng/L, 16 ng/L, 30 ng/L, 60 ng/L | 7 ng/L, 10 ng/L, 15 ng/L, 20 ng/L, 50 ng/L |
| COMPASS-MI algorithm | 4 ng/L, 8 ng/L, 40 ng/L, 60 ng/L | 7 ng/L, 11 ng/L, 55 ng/L, 70 ng/L |
| Clinical Chemistry Score | 4 ng/L, 14 ng/L, 30 ng/L (all hs-cTnI assays) | 8 ng/L, 18 ng/L, 30 ng/L (all hs-cTnT assays) |
| High-STEACS | 5 ng/L, 9 ng/, 16 ng/L, 34 ng/L | 6 ng/L, 10 ng/L, 14 ng/L, 22 ng/L |
| Perioperative monitoring and | 6 ng/L, 10 ng/L, 15 ng/L, 30 ng/L, 60 ng/L, | 7 ng/L, 10 ng/L, 14 ng/L, 20 ng/L, 65 ng/L, |
| Out-patient setting | 700 ng/L, 2600 ng/L | 1000 ng/L, 2800 ng/L |
| Laboratory compliant | 8 ng/L, 18 ng/L, 36 ng/L, 300 ng/L | 8 ng/L, 18 ng/L, 50 ng/L, 100 ng/L |
| Machine Learning Artificial | 8 ng/L, 16 ng/L, 30 ng/L, 60 ng/L, 100 ng/L, | 8 ng/L, 16 ng/L, 30 ng/L, 60 ng/L, 100 ng/L, |
| Intelligence algorithms | 200 ng/L (all hs-cTnI assays) | 200 ng/L (all hs-cTnT assays) |

While the present application has been described with reference to examples (see FIG. 1), it is to be understood that the scope of the claims should not be limited by the embodiments set forth in the examples but should be given the broadest interpretation consistent with the description.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

1. Kavsak P A. Clin Biochem. 2014 February; 47(3):147-9. doi: 10.1016/j.clinbiochem.2014.01.017. PMID: 24528890.
2. Kavsak et al. Clin Chim Acta. 2014 Sep. 25; 436:273-5. doi: 10.1016/j.cca.2014.06.014. Epub 2014 Jun. 24. PMID: 24972004.
3. Kavsak et al. Clin Chem. 2016 June; 62(6):887-9. doi: 10.1373/clinchem.2016.255448. Epub 2016 Apr. 26. PMID: 27117472.
4. Kavsak et al. Clin Chem. 2017 May; 63(5):1043-1045. doi: 10.1373/clinchem.2017.271361. Epub 2017 Mar. 21. PMID: 28325814.
5. Kavsak et al. Can J Cardiol. 2018 February; 34(2):209.e5-209.e6. doi: 10.1016/j.cjca.2017.11.010. Epub 2017 Nov. 22. PMID: 29287942.
6. Raizman et al. Clin Chim Acta. 2018 November; 486:219-220.
7. Kavsak et al. Clin Chim Acta. 2019 February; 489:58-60. doi: 10.1016/j.cca.2018.11.031. Epub 2018 Nov. 28. PMID: 30502326.
8. Kavsak et al. Clin Chim Acta. 2019 November; 498:27-29.
9. Kavsak P A, Clark L. J Appl Lab Med. 2020 Sep. 1; 5(5):1122-1124.
10. Kavsak et al. CMAJ Open. 2020 Nov. 2; 8(4):E676-E684. doi: 10.9778/cmajo.20200047. PMID: 33139388; PMCID: PMC7608943.
11. Nouri, et al. Multisite Imprecision for the Abbott, Beckman, Roche and Siemens High-Sensitivity Cardiac Troponin Assays at the Female 99TH-Percenitle: A Prospective Analytical Sub-Study From the CODE-MI Trial. 74th AACC Annual Scientific Meeting, Chicago IL, US, Wednesday, Jul. 27, 2022.
12. Kavsak et al. CMAJ. 2018 Aug. 20:190(33):E974-E984. Doi: 10.1503/cmaj. 180144.
Deveraux et al. N Engl J Med. 2022 Mar. 3; 386(9):827-836.)
Duceppe et al. J. Am. Coll. Cardiol. 2020, 75 (Suppl. 1), 110

1. A method for the preparation of a cardiac troponin quality control sample of a mammal to determine the accuracy and/or precision of a cardiac troponin assay comprising the steps of:
i) preparing a cardiac troponin concentrate from a pool of biological samples obtained from a population of the mammal having a troponin concentration of greater than 75% of an upper analytical limit of the assay;
ii) obtaining a cardiac troponin base material comprising whole blood, plasma or serum of the mammal which is interference-free and disease-free; and
iii) combining a quantity of the cardiac troponin concentrate with the cardiac troponin base material to yield a cardiac troponin quality control sample having a target concentration.

2. The method of claim 1, wherein the cardiac troponin assay is a high sensitivity assay.

3. The method of claim 1, wherein the cardiac troponin is cardiac troponin I or cardiac troponin T.

4. The method of claim 1, wherein the biological samples have a troponin concentration of 80-96% of the upper analytical limit of the cardiac troponin assay.

5. The method of claim 1, wherein the cardiac troponin is cardiac troponin I (cTnI) and the concentration of cTnI in the biological samples is at least about 20,000 ng/L.

6. The method of claim 1, wherein the cardiac troponin is cardiac troponin T (cTnT) and the concentration of cTnT in the biological samples is at least about 8000-9600 ng/L.

7. The method of claim 1, wherein the cardiac troponin concentrate is processed to remove interferences therefrom.

8. The method of claim 7, wherein the interferences include cellular debris, macrocomplexes and/or infectious agents.

9. The method of claim 1, wherein the biological sample is selected from blood, serum and plasma.

10. The method of claim 1, wherein the mammal is a human.

11. The method of claim 10, wherein the base material is derived from Type AB blood.

12. A cardiac troponin quality control sample prepared by the method of claim 1.

13. A kit comprising two or more cardiac troponin quality control samples for use to determine the accuracy and/or precision of a cardiac troponin assay, wherein the samples are prepared by the method of claim 1.

14. The kit of claim 13, comprising a lower limit of detection quality control sample having a target concentration of about 2 ng/L greater than the lower limit of detection of the cardiac troponin assay, and comprising one or more of:
i) a quality control sample which is about 4 ng/L greater than the lower limit of detection quality control sample;
ii) an upper limit of normal quality control sample at about the 99^{th} percentile of a normal population;
iii) a quality control sample having a target concentration that is at a sex-specific cut-off for cardiac myocardial infarction diagnosis;
iv) one or more quality control samples having a target concentration that define myocardial infarction risk stratification cutoff concentrations;
v) a quality control sample having a target concentration that is prognostic in non-cardiac surgery; and
vi) a quality control sample having a target concentration that is prognostic in cardiac surgery.

15. The kit of claim 13, wherein the quality control samples are for use in a high sensitivity cardiac troponin I assay and have target concentrations selected at least from:
i) 6 ng/L;
ii) 4 ng/L, 8 ng/L, 40 ng/L and 60 ng/L for use in a COMPASS-MI algorithm;
iii) 5 ng/L, 9 ng/L, 16 ng/L and 34 ng/L for use in a High-Sensitivity Troponin in the Evaluation of patients with Acute Coronary Syndrome (High-STEACS) algorithm;
iv) 4 ng/L, 6 ng/L, 16 ng/L, 30 ng/L and 60 ng/L for use in a 0/1 hour, 0/2 hour and/or 0/3 hour algorithm;
v) 4 ng/L, 14 ng/L and 30 ng/L for use with an algorithm which determines troponin levels in combination with glucose and estimated glomerular filtration rate;
vi) 6 ng/L, 10 ng/L, 15 ng/L, 30 ng/L, 60 ng/L, 700 ng/L and 2600 ng/L for use in a perioperative or out-patient setting;
vii) 8 ng/L, 18 ng/L, 36 ng/L and 300 ng/L for use in a laboratory setting; and
viii) 8 ng/L, 16 ng/L, 30 ng/L, 60 ng/L, 100 ng/L and 200 ng/L for use with machine learning algorithms;
wherein the target concentrations may vary by 1.6 ng/L or 20%.

16. The kit of claim 13, wherein the quality control samples are for use in a high sensitivity cardiac troponin T assay and have target concentrations selected from:
i) 8 ng/L;
ii) 7 ng/L, 11 ng/L, 55 ng/L and 70 ng/L for use in a COMPASS-MI algorithm;
iii) 6 ng/L, 10 ng/L, 14 ng/L and 22 ng/L for use in a High-Sensitivity Troponin in the Evaluation of patients with Acute Coronary Syndrome (High-STEACS) algorithm;
iv) 7 ng/L, 10 ng/L, 15 ng/L, 20 ng/L and 50 ng/L for use in a 0/1 hour, 0/2 hour and/or 0/3 hour algorithm;
v) 8 ng/L, 18 ng/L and 30 ng/L for use with an algorithm which determines troponin levels in combination with glucose and estimated glomerular filtration rate;
vi) 7 ng/L, 10 ng/L, 14 ng/L, 20 ng/L, 65 ng/L, 1000 ng/L and 2800 ng/L for use in a perioperative or out-patient setting;
vii) 8 ng/L, 18 ng/L, 50 ng/L and 100 ng/L for use in a laboratory setting; and
viii) 8 ng/L, 16 ng/L, 30 ng/L, 60 ng/L, 100 ng/L and 200 ng/L for use with machine learning algorithms;
wherein the target concentrations may vary by 1.6 ng/L or 20%.

17. The kit of claim 15, comprising quality control samples for use in a COMPASS-MI algorithm or a High-STEACS algorithm.

18. (canceled)

19. The kit of claim 15 or 16, comprising quality control samples for use in a 0/1 hour, 0/2 hour and/or 0/3 hour algorithm.

20. The kit of claim 15 or 16, comprising quality control samples for use in an algorithm which determines troponin levels in combination with glucose and estimated glomerular filtration rate.

21. The kit of claim 15 or 16, comprising quality control samples for use in a perioperative or out-patient setting.

22. The kit of claim 15 or 16, comprising quality control samples for use in a laboratory setting.

23. The kit of claim 15 or 16, comprising quality control samples for use with machine learning algorithms.

24. (canceled)
